(19) United States
(12) Patent Application Publication
(10) Pub. No.: US 2023/0288431 A1
(43) Pub. Date: Sep. 14, 2023

(54) USE OF MICRO-RNA TO IMPROVE AND TREAT CHRONIC PHASE CARDIAC FUNCTION

(71) Applicant: NATIONAL UNIVERSITY CORPORATION TOKAI NATIONAL HIGHER EDUCATION AND RESEARCH SYSTEM, Nagoya-shi, Aichi (JP)

(72) Inventors: Shinya MINATOGUCHI, Gifu-shi (JP); Yukihiro AKAO, Gifu-shi (JP)

(73) Assignee: NATIONAL UNIVERSITY CORPORATION TOKAI NATIONAL HIGHER EDUCATION AND RESEARCH SYSTEM, Nagoya-shi, Aichi (JP)

(21) Appl. No.: 18/009,069

(22) PCT Filed: Mar. 17, 2021
(86) PCT No.: PCT/JP2021/010808

§ 371 Date: Dec. 8, 2022

(30) Foreign Application Priority Data
Jun. 8, 2020 (JP) 2020-099660

(51) Int. Cl. G01N 33/68; A61K 31/7105; A61P 9/10

(52) U.S. Cl. CPC G01N 33/6893; A61K 31/7105; A61P 9/10; G01N 2800/324

(57) ABSTRACT

A method for assessing a chronic phase cardiac function in acute myocardial infarction, includes monitoring blood concentration of miR-143 and/or miR-145 in an acute phase of the acute myocardial infarction in an individual who has occurred the acute myocardial infarction or presents a possibility of occurrence of the acute myocardial infarction.

### TECHNICAL FIELD

### Cross Reference to Related Application

This application claims priority to Japanese Patent Application No. 2020-99660 filed on Jun. 8, 2020, the contents of which are hereby incorporated by reference into the present application.

The present teaching relates to the use of a microRNA to improve and treat chronic phase cardiac function.

### BACKGROUND ART

Heart disease is a cause of death with a high mortality rate in every country, including Japan. Acute myocardial infarction is a heart disease known to occur in 70,000 people a year also in Japan. In acute myocardial infarction, a loss of myocardium results in the occurrence of a reduction in cardiac function, which may be followed by left ventricular wall thinning and lumen enlargement and the occurrence of left ventricular enlargement, i.e., left ventricular remodeling. The progression of left ventricular remodeling leads to heart failure and a poor life prognosis.

In this context, microRNA-145-5p (miR-145 in the following) and microRNA-143-3p (miR-143 in the following) are known to suppress the proliferation of vascular smooth muscle. We have already reported that in animal experiments miR-145 is effective for myocardial tissue repair after acute myocardial infarction (Non Patent Literature 1).

### SUMMARY OF INVENTION

### Technical Problem

However, it remains unclear as to whether endogenous miR-145 and miR-143 are mobilized into the bloodstream after acute myocardial infarction. It is also unclear as to whether mobilized endogenous miR-145 and miR-143 improve chronic phase cardiac function. As noted above, acute myocardial infarction has a poor life prognosis. To date, it has been difficult to predict reductions in chronic phase cardiac function in acute myocardial infarction patients. Preventing deterioration in chronic phase cardiac function and treatment in an early stage are also desirable.

The present teaching provides art that, while in the acute phase after acute myocardial infarction, predicts chronic phase cardiac function and ameliorates reductions in cardiac function.

### Solution to Technical Problem

Upon investigating whether miR-145 and miR-143 are mobilized into the blood of a patient after acute myocardial infarction, the present inventors discovered for the first time a correlation between increases in the amount of this mobilization and improvement in chronic phase cardiac function. Moreover, a suppression of reductions in cardiac function was confirmed when an evaluation was performed as to whether reductions in chronic phase cardiac function could be ameliorated by the administration of such microRNAs. The following means are provided based on the findings.
[1] A method for assessing a chronic phase cardiac function in acute myocardial infarction, comprising monitoring blood concentration of miR-143 and/or miR-145 in an acute phase of the acute myocardial infarction in an individual who has occurred the acute myocardial infarction or presents a possibility of occurrence of the acute myocardial infarction.
[2] The method according to [1], wherein the monitoring is performed in any interval within two weeks from immediately after the occurrence of the acute myocardial infarction in the individual.
[3] The method according to [1] or [2], wherein the monitoring estimates chronic phase cardiac function of the individual based on an amount of change over time in the blood concentration.
[4] The method according to any one of [1] to [3], wherein the monitoring estimates a favorable chronic phase cardiac function for the individual when the blood concentration increases over time.
[5] The method according to any one of [1] to [4], wherein the monitoring estimates a poor chronic phase cardiac function for the individual when the blood concentration does not increase over time.
[6] The method according to any one of [1] to [5], wherein the monitoring compares the amount of change over time in the blood concentration with a reference value that can estimate the chronic phase cardiac function of the individual.
[7] An apparatus of predicting a chronic phase cardiac function for an acute myocardial infarction, the apparatus being configured to acquire an amount of change over time in an acute phase blood concentration of miR-143 and/or miR-145 acquired for an individual who has occurred the acute myocardial infarction or presents a possibility of occurrence of the acute myocardial infarction.
[8] The apparatus according to [7], wherein the apparatus predicts the chronic phase cardiac function by comparing the amount of change over time with a preliminarily acquired reference value that can predict the chronic phase cardiac function of the individual.
[9] A system of predicting a chronic phase cardiac function for an acute myocardial infarction, the system comprising a means of acquiring an amount of change over time in acute phase blood concentration of miR-143 and/or miR-145 acquired for an individual who has occurred the acute myocardial infarction or presents a possibility of occurrence of the acute myocardial infarction.
[10] The system according to [9], the system further comprising a means of performing a comparison of the amount of change over time with a preliminarily acquired reference value that can predict the chronic phase cardiac function of the individual and that performs a prediction of the chronic phase cardiac function, based on results of the comparison.
[11] A drug for improving or treating a chronic phase cardiac function after acute myocardial infarction, comprising miR-143 and/or miR-145 or a compound that acts like miR-143 and/or miR-145 in an individual as an active ingredient thereof.
[12] The drug according to [11], that is administered in an acute phase after an acute myocardial infarction.
[13] A drug for improving or treating acute myocardial infarction, comprising miR-143 and/or miR-145 or a compound that acts like miR-143 and/or miR-145 in an individual as an active ingredient thereof.
[14] A test kit of predicting a chronic phase cardiac function after acute myocardial infarction, wherein the kit comprises a reagent that specifically detects miR-143 and/or miR-145.

### BRIEF DESCRIPTION OF DRAWINGS

FIG. 1 contains diagrams that respectively show, in accordance with the examples, (a) the plasma concentration of miR-145 on day 0 and day 7 after acute myocardial infarction and (b) the plasma concentration on day 0 and day 7 of miR-143 after acute myocardial infarction;

FIG. 2 contains, in accordance with the examples, a diagram (a) that shows the correlation between the amount of change in the plasma concentration of miR-145 between day 0 and day 7 after acute myocardial infarction and the amount of change in the EF (ΔEF %) between the time of onset and after six months, and a diagram (b) that shows the correlation between the amount of change in the plasma concentration of miR-143 between day 0 and day 7 after acute myocardial infarction and the amount of change in the EF (ΔEF %) between the time of onset and after six months;

FIG. 3 shows the effect of miR-143 on cardiac function in a rat myocardial infarction model, wherein diagram (a) shows the left ventricular ejection fraction (LVEF) % by echocardiogram after the administration of miR-143 and the left ventricular fractional shortening (LVFS) % by echocardiogram after the administration of miR-143, and diagram (b) provides echocardiogram images;

FIG. 4 shows the effect on the viable cell count in rat H9C2 cardiomyocytes due to exposure to hydrogen peroxide, where diagram (a) shows the viable cell count for the cardiomyocytes 3 days after exposure to hydrogen peroxide and diagram (b) shows the inhibitory effect, due to transfection with miR-143 #12, on oxidative stress in H9C2 cells induced by exposure to hydrogen peroxide (the viable cell count is shown at 5 days after the cardiomyocytes were transfected with control miRNA or miR-143 #12 immediately after exposure to aqueous hydrogen peroxide);

FIG. 5 contains a diagram (a) that shows the number of cardiomyocyte ropes formed after exposure of rat H9C2 cardiomyocytes to hydrogen peroxide, and a diagram (b) that shows the shape of the cardiomyocyte ropes that are formed, and
(a) is a graph that shows the number of cell ropes after transfection with miR-143 #12, and (b) is a diagram that shows, bounded by a circle, the presentation under a phase-contrast microscope of a cardiomyocyte rope;

FIG. 6 shows the effects (heart section observation and infarct size) of the administration of miR-143 #12 to a rat acute myocardial infarction model (reperfusion for 14 days after occlusion of the coronary artery for 30 minutes);

FIG. 7 contains a diagram (A) that shows the number of CD31-positive microvessels due to the intravenous injection of miR-143 #12 in a rat myocardial infarction model (reperfusion for 14 days after occlusion of the coronary artery for 30 minutes), and a diagram (B) that shows the number of alpha-smooth muscle-positive vessels having vascular smooth muscle;

FIG. 8 shows the reduction in the number of TUNEL-positive myocytes due to the systemic administration of miR-143 #12 to a rat myocardial infarction model (reperfusion for 14 days after occlusion of the coronary artery for 30 minutes);

FIG. 9 shows the increase in the number of ki67-positive cardiomyocytes in the myocardial infarct border zone after the administration of miR-143 #12 in a rat myocardial infarction model (reperfusion for 14 days after occlusion of the coronary artery for 30 minutes);

FIG. 10 shows the effect of the introduction of miR-143 #12 on HUVEC (human umbilical vein endothelial cell) tube formation;

FIG. 11 shows the results of the introduction of 7 miR143 derivatives and A-miR143 (purchased from Ambion, Inc.) after exposing rat H9C2 cardiomyocytes to hydrogen peroxide (40 μM) for 30 minutes; and

FIG. 12 shows the constitution of the miR-143 derivatives used in Example 7.

### DESCRIPTION OF EMBODIMENTS

The disclosure in the present teachings relate to art that, while in the acute phase after acute myocardial infarction, predicts chronic phase cardiac function and ameliorates reductions in cardiac function. More particularly, the disclosure in the present teaching relates to art for predicting, in a very early phase, i.e., the acute phase, after acute myocardial infarction, the subsequent chronic phase cardiac function, and relates to art for predicting and improving chronic phase cardiac function after acute myocardial infarction, wherein this art can improve future chronic phase cardiac function through treatment based on the prediction in this very early phase.

Based on the new knowledge—i.e., that when an individual who has occurred acute myocardial infarction has occurred or who presents the possibility of the occurrence of acute myocardial infarction (referred to hereafter simply as “the individual” or “an individual”) exhibits an increase in the amount of change over time in the acute phase blood concentration of miR-143 and/or miR-145, the chronic phase cardiac function is then recovered in proportion to this amount—the disclosure in the present teaching provides, inter alia, a method of assessment that can predict chronic phase cardiac function from the size of the amount of change over time in the acute phase. In addition, based on the new knowledge that the chronic phase cardiac function can be improved by the administration of miR-143 and/or miR-145, the disclosure in the present teaching provides a drug and a method for which an active ingredient is, inter alia, miR-143 and/or miR-145, which drug and method can prevent or treat reductions in chronic phase cardiac function.

The use of the method, etc., disclosed in the present teaching enables prediction, in a very early phase, of the chronic phase cardiac function of an individual, and thereby makes it possible to also carry out an assessment in the very early phase of whether to implement various medical treatments for improving the chronic phase cardiac function, thus enabling the effective and economical practice of medicine. In addition, the blood concentration of miR-143 and miR-145 can be readily measured, and as a consequence an effective prediction can be readily performed. Furthermore, reductions in the chronic phase cardiac function can be prevented or treated by the administration of, inter alia, miR-143 and/or miR-145, in the very early phase after acute myocardial infarction.

According to the present inventors, when the blood concentration of miR-143 and miR-145 in the acute phase after the onset of acute myocardial infarction was observed, it generally underwent an increase. However, when the increases were individually scrutinized, it was found that a larger increase was associated with a higher degree of recovery of the left ventricular ejection fraction (LVEF), which is an indicator of cardiac function in the chronic phase.

miR-143 is known to inhibit the proliferation of vascular smooth muscle, and miR-145 has been shown to be effective in myocardial tissue repair after acute phase myocardial infarction. However, it cannot be said that acute myocardial infarction and a subsequent reduction in chronic phase cardiac function have a common mechanism of onset, and as a consequence it could not have been anticipated that a component effective in acute myocardial infarction would be effective for improving chronic phase cardiac function at the same time. It could not have been predicted, even by the inventors, that the acute phase blood concentrations of such miRNAs could serve as good indicators of reductions in chronic phase cardiac function that accompany left ventricular remodeling, or that such miRNAs could also be therapeutic components for chronic phase cardiac function.

Typical and non-limiting specific examples of the disclosures of the Description are explained in detail below with reference to the drawings. These detailed explanations are aimed simply at showing preferred examples of the disclosures of the Description in detail so that they can be implemented by a person skilled in the art, and are not intended to limit the scope of the disclosures of the Description. The additional features and disclosures disclosed below may be used separately or together with other features and inventions to provide a further improved use of micro-RNA to improve and treat chronic phase cardiac function.

The combinations of features and steps disclosed in the detailed explanations below are not essential for implementing the disclosures of the Description in the broadest sense, and are presented only for purposes of explaining typical examples of the disclosures of the Description in particular. Moreover, the various features of the typical examples above and below and the various features described in the independent and dependent claims do not have to be combined in the same way as in the specific examples described here, or in the listed order, when providing addition useful embodiments of the disclosures of the Description.

All features described in the Description and/or Claims are intended as individual and independent disclosures restricting the initial disclosures and the claimed matter specifying the invention, separately from the constitution of features described in the Examples and/or Claims. Moreover, all descriptions of numerical ranges and groups or sets are intended to include intermediate configurations for purposes of restricting the initial disclosures and the claimed matter specifying the invention.

The art disclosed in the present teaching is described in detail in the following.

(The Method for Assessing Chronic Phase Cardiac Function in Acute Myocardial Infarction)

This assessment method comprises monitoring the blood concentration of miR-143 and/or miR-145 in the acute phase in an individual who has occurred acute myocardial infarction or who presents the possibility of the occurrence of acute myocardial infarction. Here, acute myocardial infarction refers to the acute occurrence of, e.g., a blockage or constriction in the coronary artery, resulting in a decline in blood flow, with the myocardium assuming an ischemic state or necrotic state. The cause of the coronary artery stenosis, occlusion, etc., is not a particular consideration here. Whether an acute myocardial infarction has occurred is generally determined by a physician based on various assessments and examinations. Individuals targeted by the present assessment method include individuals for whom a diagnosis of acute myocardial infarction has been established by a physician, as well as individuals before a diagnosis of acute myocardial infarction has been established.

The individual may be a mammal, including humans, and can be exemplified by pets such as cats, dogs, and so forth, and also by various domestic animals.

The acute phase of acute myocardial infarction generally refers to from immediately after onset to about two weeks thereafter.

The chronic phase of acute myocardial infarction begins at one month from immediately after onset. It has been reported that if the left ventricular ejection fraction (EF) is improved at 2 weeks, 6 weeks, and 8 weeks after the onset of myocardial infarction, the prognosis thereafter will be improved (Chew D S, JACC Clinical Electrophysiology 2018). In addition, measurement at 6 months is significant because evaluation of cardiac function and remodeling at 6 months can predict the prognosis thereafter (Bolognese L, Circulation 2002).

The present assessment method comprises monitoring the blood concentration of miR-143 and/or miR-145 in the acute phase of acute myocardial infarction. Specifically, this blood concentration is measured at least two different times in the acute phase.

miR-143, for example, in humans, can have a sense strand (ggugcagugc ugcaucucug gu) as described in SEQ ID NO: 1 and an antisense strand (ugagaugaag cacuguagcu c) as described in SEQ ID NO: 2, respectively. microRNA sequences can be obtained as desired, for example, from http://www.mirbase.org/, which is a database of microRNAs. miR-145, for example, in humans, can have a sense strand (guccaguuuu cccaggaauc ccu) as described in SEQ ID NO: 6 and an antisense strand (ggauuccugg aaauacuguu cu) as described in SEQ ID NO: 7, respectively. The miR-143 and miR-145 that are the identification targets for the present assessment are established as appropriate in correspondence to, for example, the species of the individual.

miRNA is a single-stranded RNA at the point of effect, but the primary miRNA that is its precursor adopts the configuration of an RNA strand having a loop structure; in addition, it becomes a double-stranded RNA, a single-stranded RNA of which becomes the miRNA. In the present assessment method, the measurement target may be the single-stranded RNA that is the final form, or the measurement target may be the other strand of the double-stranded RNA of the loop structure, but the measurement target is advantageously the single-stranded RNA of the final effective configuration.

In the present assessment method, the measurement target may be only one of either miR-143 and miR-145, or the measurement target may be both of these. For example, the measurement target may be only miR-143.

The sample for measurement of the miR-143 and/or miR-145 can be blood collected from the individual. The collection site and so forth are not particularly limited, but from the viewpoint of consistent collection, for example, the peripheral blood, which can be obtained by a general method for collecting venous blood, can be used. In addition, the blood concentration of miR-143 and/or miR-145 may be the concentration in the blood or may be the concentration in the plasma. The blood concentration of miR-143 and/or miR-145 can be measured as the plasma concentration.

The method for measuring the blood concentration of miR-143 and/or miR-145 is not particularly limited, and known methods can be used as appropriate. As a general matter, the plasma is separated from the blood, the RNA is extracted from the separated plasma, and miR-143 and/or miR-145 is quantified using this RNA extract. RNA extraction from blood is well known to those skilled in the art, and extraction can be performed using various commercially available kits based on the protocols for the kits or common procedures in this field.

Alternatively, cDNA may be obtained from the RNA extract using, for example, primers and reverse transcriptase (RNA-dependent DNA polymerase) that targets single-stranded RNA, and amplification and detection may be carried out, using prescribed primers, of the cDNA for the miR-143 or miR-145 that is the RNA target, by PCR, e.g., real-time PCR (RT-PCR), using this cDNA as template. Quantitative measurements can be performed using, for example, reverse transcription PCR, proceeding from the RNA. Well-known techniques and kits, such as one-step or two-step RT-PCR, can be used as appropriate for detection and quantification through reverse transcription of the RNA and PCR.

Monitoring of the blood concentration of miR-143 and/or miR-145 is performed during the acute phase (immediately after onset to 4 weeks) of acute myocardial infarction in the individual. According to the present inventors, during the acute phase (immediately after onset to 2 weeks) of acute myocardial infarction, in all individuals the blood concentration of miR-143 and/or miR-145 exhibits a certain trend, such as generally increasing or declining with elapsed time. On the other hand, because detection of the amount of time elapsed for a blood concentration of at least a certain level is favorable from the viewpoint of test accuracy, and because earlier diagnosis is advantageous, the length of the monitoring period is, for example, two weeks within the acute phase, and, for example, about one week within the acute phase. In addition, the monitoring period itself is within 2 weeks from immediately after onset, and, for example, within 1 week from immediately after onset.

The blood concentration of miR-143 and/or miR-145 is measured at least twice during the monitoring period. Proceeding in this manner makes it possible to readily comprehend the trend of increase and the amount of change overtime in the blood concentration during the monitoring period. For example, measurement at the beginning and end of a monitoring period enables convenient acquisition of the maximum amount of change over time during this period. When measurement is performed three or more times within the monitoring period, the amount of change overtime is then acquired from the first blood concentration and the last blood concentration within this period.

The data on the blood concentrations of miR-143 and/or miR-145 and their amounts of change over time need not be expressed in units based on the mass of the miR-143 and/or miR-145, such as μg/mL, μmol/mL, and so forth. This data may be data that can be related to the blood concentration of miR-143 and/or miR-145, such as the signal intensity in PCR or, for example, the signal intensity ratio to an internal indicator, but is otherwise not particularly limited.

In addition, the amount of change over time in the miR-143 and/or miR-145 blood concentration, for example, may be the difference between the last blood concentration and the first blood concentration during the monitoring period, or may be the ratio of the earliest blood concentration to the final blood concentration.

The monitoring of the miR-143 and/or miR-145 blood concentration enables acquisition of the amount of change over time in these concentrations and enables estimation of the chronic phase cardiac function of an individual based on this amount of change over time. When the blood concentration increases with time, a good chronic phase cardiac function can be estimated for the individual. When the blood concentration does not increase with time or when it decreases with time, a poor chronic phase cardiac function can be estimated for the individual.

It can be said that the larger the amount of change over time in this blood concentration, the greater the trend of improvement in the chronic phase cardiac function, and recovery of or improvement in the chronic phase cardiac function can be affirmed. In addition, a smaller amount of change corresponds to a declining trend for chronic phase cardiac function, and a reduction in chronic phase cardiac function can be affirmed. Thus, even when the amount of change over time presents an increasing trend, if the amount of the change (amount of increase) is small, there may be instances when a poor chronic phase cardiac function can be estimated rather than being able to estimate a good chronic phase cardiac function. Here, the chronic phase cardiac function can be exemplified by the left ventricular ejection fraction (LVEF, %) and left ventricular fractional shortening (LVFS, %) as well as by other indicators of cardiac function, e.g., the left ventricular end-diastolic diameter (LVDd), left ventricular end-systolic diameter (LVDs), left ventricular end-diastolic volume (LVEDV), left ventricular end-systolic volume (LVESV), +dP/dt, and −dP/dt.

In order to predict chronic phase cardiac function based on the amount of change over time, it is advantageous to preliminarily establish a reference value that enables the prediction of chronic phase cardiac function based on the amount of change over time in the aforementioned blood concentrations and an amount of change over time that functions as an indicator of chronic phase cardiac function, and to carry out a comparison with this reference value. For example, while acquiring the amount of change over time for a plurality of individuals, the amount of change over time in data (for example, differences in and/or ratios of, e.g., LVEF, LVFS, LVDd, LVDs, LVEDV, LVESV, +dP/dt, and −dP/dt, that function as indicators of ventricular remodeling) that functions as an indicator of cardiac function in these individuals may also be acquired. Since the amount of change over time in cardiac function is an indicator of chronic phase cardiac function, it is preferable to use the amount of change over time between the acute phase after the onset of acute myocardial infarction and the chronic phase (from 6 months after onset). The time period for measuring cardiac function in order to calculate the amount of change over time is not particularly limited, but, for example, the amount of change between the LVEF immediately after the onset of acute myocardial infarction and the LVEF at the 6-month time point for the chronic phase can be used.

Just as with the miR-143 and/or miR-145 blood concentration, the amount of change over time in cardiac function may be a difference or a ratio. When a difference is used for the amount of change over time in the blood concentration, the same method can be used to calculate the amount of change over time, e.g., the amount of change over time for cardiac function may also be a difference. The amount of change over time in cardiac function and the reference value may also be associated with an indicator of cardiac function, and their unit amount and so forth are not particularly limited.

By comparing the amount of change over time in blood concentration with the amount of change over time in cardiac function, a reference value for the amount of change over time in blood concentration, which is associated with a prescribed amount of change over time in cardiac function, can be established. Those skilled in the art can establish such a reference value as appropriate based on known statistical methods. For example, an indicator that can affirm improvement in chronic phase cardiac function is, for example, a difference in LVEF between the acute phase and the chronic phase of more than 0%; or, for example, at least 1%; or, for example, at least 5%; or, for example, at least 10%; or, for example, at least 15%; or, for example, at least 20%. To the contrary, an indicator that can affirm a deterioration in chronic phase cardiac function is a difference in LVEF between the acute phase and the chronic phase, for example, of not more than 0%; or, for example, not more than 5%; or, for example, not more than 10%. Those skilled in the art can obtain such reference values using various statistical methods as appropriate.

For example, when the monitoring period in the acute phase is from immediately after onset to one week, and when the concentration ratio of miR-143 and miR-145, respectively, to miR-16 is used as the blood concentration and a difference is used for the amount of change over time in blood concentration, the maintenance or recovery of chronic phase cardiac function can be predicted when this amount of change over time is, for example, at least 0.05. Or, it is, for example, at least 0.06; or, for example, at least 0.07; or, for example, at least 0.08; or, for example, at least 0.09; or, for example, at least 0.10; or, for example, at least 0.11; or, for example, at least 0.12; or, for example, at least 0.13; or, for example, at least 0.14; or, example, at least 0.15. Further recovery of chronic phase cardiac function can be predicted when this amount of change over time is at least 0.7.

In addition, for example, a decline in chronic phase cardiac function can be predicted when the aforementioned amount of change over time is less than 0.05. Or, when, for example, this amount is not more than 0.04; or, for example, not more than 0.03; or, for example, not more than 0.02; or, for example, not more than 0.01.

As has been described in the preceding, the acute phase monitoring of miR-143 and/or miR-145 in accordance with the present assessment method makes it possible to predict chronic phase cardiac function from the corresponding the change over time. In addition, the degree of improvement or degree of deterioration in chronic phase cardiac function can also be predicted from the size of the amount of change over time.

(Method of Establishing a Reference Value that Predicts Chronic Cardiac Function)

According to the present teaching, an aspect of the teaching in the present teaching is also a method of establishing a reference value that is used in the present assessment method and that can predict chronic cardiac function. This method of establishing a reference value makes it possible to more conveniently make a highly precise and highly accurate prediction of chronic phase cardiac function.

(Test Kit for Predicting Chronic Cardiac Function)

Also provided in accordance with the present teaching is a test kit for predicting chronic phase cardiac function during the acute phase of acute myocardial infarction. This kit can comprise the reagents necessary for measuring the blood concentrations of miR-143 and/or miR-145. Such reagents can be exemplified by primer sets for the specific reverse transcription of miR-143 and/or miR-145. Other examples are primer sets for specifically amplifying the cDNA of the miR-143 and/or miR-145 from cDNA obtained from RNA in the blood of an individual. Reagents for extracting RNA, RNA-dependent DNA polymerase for reverse transcription, DNA polymerase for PCR, nucleotides, buffers, and so forth may as necessary also be included. Such a kit may be configured as a kit for performing real-time PCR.

(Apparatus Used to Predict Chronic Phase Cardiac Function for Acute Myocardial Infarction)

The apparatus disclosed in the present teaching is configured to acquire the amount of change over time in the acute phase blood concentration of miR-143 and/or miR-145, that is acquired for an individual in whom acute myocardial infarction has occurred or who presents the possibility of the occurrence of acute myocardial infarction. The acquisition of this amount of change over time using the present apparatus can contribute to prediction of the chronic phase cardiac function for an acute myocardial infarction. The present apparatus is basically a computer comprising a storage means, e.g., various memories, capable of storing this amount of change over time as data, and comprising a control means, e.g., a CPU, that can use the amount of change over time stored in the storage means, by the input/output of same as appropriate, for calculations. The data relating to the amount of change over time may be input from the apparatus for measuring the data to the storage means via an input/output interface, or may optionally be input as appropriate. The storage means can also store a program that causes the CPU to execute input/output processing and arithmetic processing of this data. The present apparatus can as desired also be provided with an output means, such as a display or printer.

The present apparatus can predict chronic phase cardiac function by comparing the aforementioned amount of change over time with a preliminarily acquired reference value that can predict the chronic phase cardiac function of the individual. For this prediction, the control means compares the amount of change over time stored in the storage means with a reference value that has been input or stored in the storage means, and, when a predetermined condition is satisfied, the calculation result, i.e., that maintenance or improvement of the chronic phase cardiac function can be affirmed, is calculated and displayed. In addition, when a different predetermined condition is satisfied, the calculation result, i.e., that a deterioration in the chronic phase cardiac function can be affirmed, is calculated and displayed.

For the reference value, a preliminarily prepared reference value corresponding to, inter alia, the type of the amount of change over time, is selected. The operator of the apparatus can also input a reference value as appropriate, or can freely select a reference value from a plurality of prepared reference values. For example, the calculation result of maintenance or improvement of chronic phase cardiac function is output when—upon comparison of the amount of change over time with a reference value for affirming the maintenance or improvement of chronic phase cardiac function—the amount of change over time is greater than or equal to the reference value.

(System Used to Predict the Chronic Phase Cardiac Function of an Acute Myocardial Infarction)

The system disclosed in the present teaching can be provided with an acquisition means that acquires the amount of change over time in the acute phase blood concentration of miR-143 and/or miR-145, that is acquired for an individual in whom acute myocardial infarction has occurred or who presents the possibility of the occurrence of acute myocardial infarction. The present system can comprise, for example, a prescribed server and client computer, or a cloud, as constituent elements, and makes possible various predictions based on the acquired amount of change over time in the blood concentration. The present system comprises means for acquiring the amount of change over time and, as necessary, control means that performs a prediction of chronic phase cardiac function by comparing the amount of change over time with a reference value. All of these means can be on a prescribed server or client or in a cloud. As necessary, execution may be performed by calling the amount of change over time, as well as the reference value and an arithmetic processing program, from any constituent element. The present system can also be provided with an input means and/or an output means, e.g., a display, printer, and so forth, as desired.

The aforementioned acquisition means is a means that, as for the present apparatus, stores the amount of change over time, and may be a prescribed server, or may be a client computer, or may be in a cloud. As necessary, one or two or more types are selected or a configuration enabling selection is implemented. In addition, the control means, e.g., a CPU being a means that compares the amount of change over time with a preliminarily prepared reference value that can predict the chronic phase cardiac function of the individual, and that executes a prediction of chronic phase cardiac function based on the results of this comparison may also be a prescribed server, or may be a client computer, or may be in a cloud. As necessary, one or two or more types are selected or a configuration enabling selection is implemented. Furthermore, the program for such prediction processing may also be a prescribed server, or may be a client computer, or may be in a cloud.

(Drug for Preventing, Ameliorating, or Treating Reductions in Chronic Phase Cardiac Function after Acute Myocardial Infarction)

The drug disclosed in the present teaching can contain, as an active ingredient, miR-143 and/or miR-145 or a compound that acts like miR-143 and/or miR-145 in the individual. With the present drug, the maintenance or improvement of chronic phase cardiac function can be expected through the administration in the very early stage of such an active ingredient to the individual who has occurred acute myocardial infarction has occurred. The present drug, as a result of its administration in the acute phase, can prevent, ameliorate, or treat a deterioration in cardiac function subsequent thereto. As a consequence, the present drug can be said to also be a drug for improving or treating acute myocardial infarction.

The present drug is not necessarily a drug that is to be administered only when a deterioration in chronic cardiac function is predicted based on the present assessment method, nor is it a drug that is to be administered only during the acute phase of acute myocardial infarction. It can be used from the acute phase into the chronic phase as appropriate for the prevention, improvement, and treatment of reductions in chronic phase cardiac function.

Besides miR-143 and/or miR-145, the present drug may have as an active ingredient a compound that acts like miR-143 and/or miR-145 (hereinafter referred to simply as an analog or analogs). For example, analogs of miR-143 can be exemplified by the derivatives of miR-143 disclosed in, for example, Japanese Patent Application Laid-open No. 2011-251912 and WO 2017/179660, and analogs of miR-145 can be exemplified by the derivatives of miR-145 disclosed in WO 2018/079841. These derivatives have improved in vivo stability and improved effects as microRNAs and are expected to have greater effects than natural miR-143 and/or miR-145.

In the miR-143 derivatives disclosed in WO 2017/179660, for example, a first strand is an oligonucleotide comprising the sequence described in SEQ ID NO: 3 or a sequence obtained by the substitution, deletion, insertion, or addition of 1 or 2 bases in the sequence described in SEQ ID NO: 3, which does not have a 3′-terminal modification and may have a 5′-terminal modification, and a second strand is an oligonucleotide comprising the sequence described in SEQ ID NO: 4 or a sequence obtained by the substitution, deletion, or insertion of 1 or 2 bases in the sequence described in SEQ ID NO: 4, which may have a 3′-terminal modification and may have a 5′-terminal modification, or is an oligonucleotide comprising the sequence described in SEQ ID NO: 5 or a sequence obtained by the substitution or insertion of 1 or 2 bases in the sequence described in SEQ ID NO: 5, which does not have a 3′-terminal modification and may have a 5′-terminal modification. The 3′-terminal modification is a 1- to 5-mer oligonucleotide derivative possibly containing a nucleoside derivative and/or a modified internucleoside linkage, or is a benzene-pyridine derivative. The 5′-terminal modification is a phosphate ester moiety or a group represented by the formula ═CQ₁-P(═O)(OH)₂ (in the formula, Qi is hydrogen, a halogen, substituted or unsubstituted alkyl, substituted or unsubstituted alkenyl, substituted or unsubstituted alkynyl, or substituted or unsubstituted alkyloxy). For example, the miR-143 derivative is an oligonucleotide derivative in which (A) a first strand is an oligonucleotide comprising the sequence described in SEQ ID NO: 3 and a second strand is an oligonucleotide comprising the sequence described in SEQ ID NO: 4 or 5; (B) the nucleoside derivative is (C) a nucleoside having a substituent at the 2′-position of the sugar or a nucleoside having a bridge structure between the 4′- and 2′-positions of the sugar; (D) the substituent is F, OCH₃, or OCH₂CH₂OCH₃; (E) the bridge structure is 4′-(CH₂)m-O-2′ (m is an integer from 1 to 4) or 4′-C(═O)—NR³-2′ (R³ is a hydrogen atom or alkyl); and/or (F) the modified internucleoside linkage is a phosphorothioate linkage.

An example is an miR-143 derivative in which the first strand may be an RNA oligonucleotide in which the internucleoside linkages are phosphodiester bonds, the second strand may be an oligonucleotide containing a nucleoside derivative and/or a modified internucleoside linkage, and a group represented by the formula dX¹dX² (where X¹ or X² is each independently A, G, C, or T) or a benzene-pyridine derivative may be bonded to the 3′-terminal of the second strand, wherein this derivative may have any of, or two or more of, the aforementioned (A) to (F). It may be additionally provided with any of, or with a combination of two or more of, the following characteristic features: (H) a phosphate ester moiety is bonded to the 5′-terminal of the second strand; (I) X¹ and X² are T; and (J) the first strand is an oligonucleotide having 30 or fewer bases, that contains the sequence described by SEQ ID NO: 1 or 3 or contains a sequence provided by the substitution, insertion, deletion, and/or addition of one or a plurality of bases in the sequence described by SEQ ID NO: 1 or 3, and the second strand is an oligonucleotide having 30 or fewer bases, that contains the sequence described in SEQ ID NO: 2 or 4 or contains a sequence provided by the substitution, insertion, deletion, and/or addition of one or a plurality of bases in the sequence described by SEQ ID NO: 2 or 4.

Those skilled in the art can as appropriate produce miR-143 and/or miR-145 and their analogs based on WO 2017/179660.

Any formulation and method of administration known for miRNAs in this field may be used for the formulation and method of administration of the present drug. Examples of the formulation and method of administration of miRNAs are also disclosed in the following publications: Nature Review Drug Discovery, 13, 622-638 (2014); Non Patent Literature 1; WO 2010/050328; WO 2011/064130; WO 2011/153542; WO 2013/163258; and WO 2013/192486.

The present drug may be administered to the heart topically or systemically, with there being no particular limitation, and administration may be carried out using various methods. The method of administration can be exemplified by topical administration; percutaneous administration via a catheter; intravenous injection or infusion; subcutaneous, intraperitoneal, or intramuscular injection; and pulmonary administration by aspiration or inhalation. Intravenous injection or subcutaneous administration is preferred.

The type of formulation for the topical administration of the present drug is not particularly limited, and, for example, formulations such as patches and liquids can be used. Additional examples are sterile suspensions and solutions provided by dissolution in water, buffers, diluents, or non-aqueous media.

In order to promote the introduction into the target cells of the miR-143 and/or miR-145 or analogs thereof that are the active ingredient, the present drug can contain a nucleic acid transfection reagent. Known transfection reagents can be used as this nucleic acid transfection reagent, e.g., atelocollagen; liposomes; nanoparticles; and cationic lipids such as Lipofectin, Lipofectamine, DOGS (Transfectam), DOPE, DOTAP, DDAB, DHDEAB, HDEAB, Polybrene, and poly(ethyleneimine) (PEI).

The administration of the present drug is established as appropriate based on the prediction of chronic phase cardiac function, other assessments, and diagnostic information. For example, a course of treatment can continue for from one day to several months, or until recuperation is realized or until remission of the condition is achieved. Optimal dosing schedules can be calculated from measurement of drug accumulation in vivo. The optimal dosage, method of administration, and repetition frequency can be determined by those skilled in the art in this field. The optimal dosage will vary depending on the relative potency of the individual active ingredients, i.e., miR-143 and/or miR-145 or their analogs, but can generally be calculated based on the IC₅₀ or EC₅₀ in vitro and from in vivo animal experiments. For example, when the molecular weight of the miR-143 derivative (derived from the sequence and chemical structure of the miR-143 derivative) and the (experimentally derived) effective dose, such as the IC₅₀, are available, the dosage in mg/kg may be calculated as a matter of routine.

### EXAMPLES

Examples that realize the embodiments disclosed in the present teaching are described in the following. However, the disclosure of the present teaching is not limited to or by the following examples.

### Example 1

(Elucidation of the Correlation Between ΔLVEF and ΔmiR-143 and ΔmiR-145 in Human Acute Myocardial Infarction Patients)

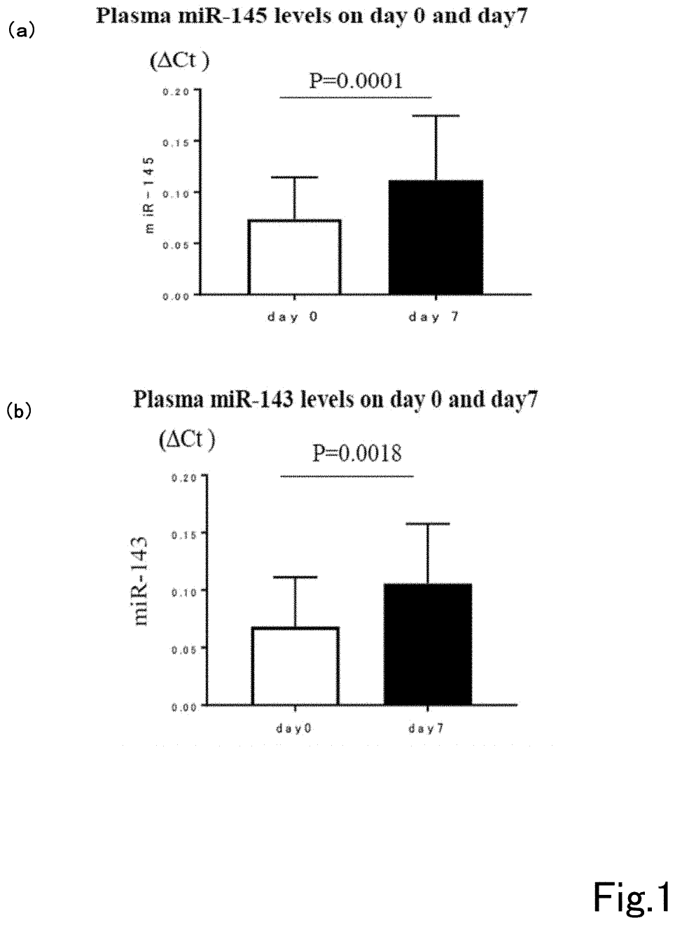

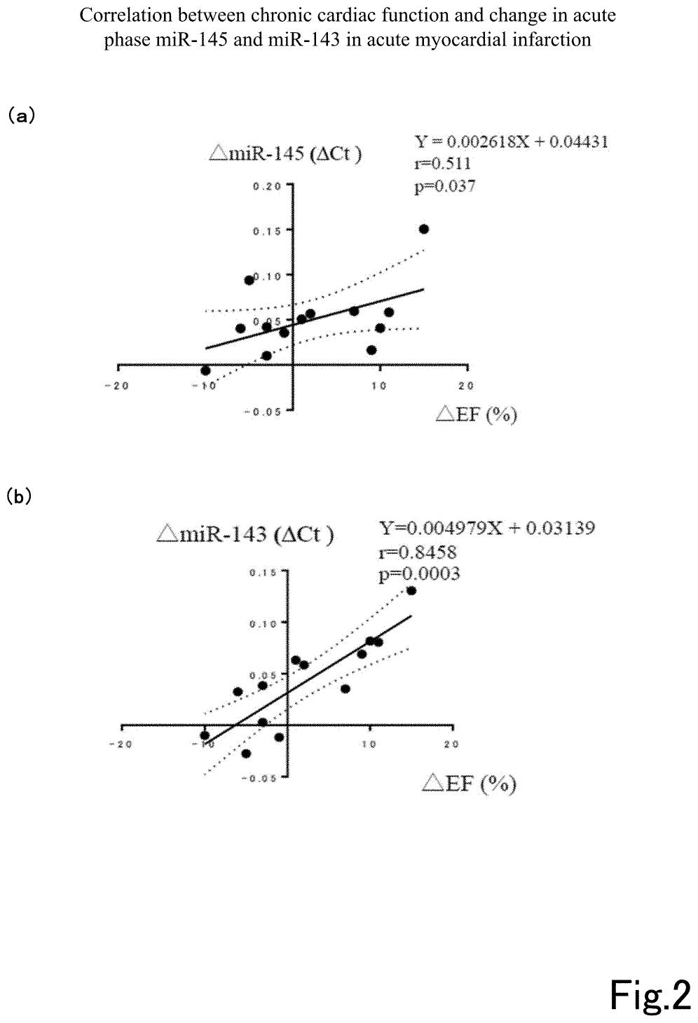

For a group of 23 human patients (AMI) diagnosed with acute myocardial infarction or possible acute myocardial infarction, venous blood collection was performed on the day of admission (day 0), after 1 day (day 1), and after 1 week (day 7) after the occurrence of acute myocardial infarction. The total RNA was isolated using a NucleoSpin miRNA isolation kit, and qRT-PCR was performed using a TaqMan microRNA assay and THUNDERBIRD Probe qPCR Mix. The plasma miR-145 and miR-143 levels on day 0 and day 7 were determined as the ratio with microRNA-16 used as the internal indicator. For these same cases, ΔmiR-145 and ΔmiR-143 were defined by subtracting miR-145 and miR-143 on day 0 from miR-145 and miR-143 on day 7, respectively. ΔmiR-145 and ΔmiR-143 for all the patients tested are shown in FIG. 1. In addition, the left ventricular ejection fraction (LVEF) was measured using echocardiography at the time of admission and 6 months later, and the difference (ΔLVEF) between the LVEF at the time of admission and 6 months later was determined. The correlations (n=13) between ΔmiR-145 and ΔLVEF and between ΔmiR-143 and ΔLVEF for all the patients test were determined. The results are shown in FIG. 2.

As shown in FIG. 1, for both miR-145 and miR-143 there was a significant increase at day 7 versus day 0. In addition, as shown in FIG. 2, for both ΔmiR-145 and ΔmiR-143, which were increments, a significant positive correlation with ΔLVEF was recognized.

Based on the preceding, it was found that the plasma miR-145 and miR-143 in the early phase of acute myocardial infarction can serve as an indicator that can predict improvement in the LVEF in the chronic phase.

### Example 2

(Effect of miR-143 #12 on Cardiac Function in a Rat Myocardial Infarction Model)

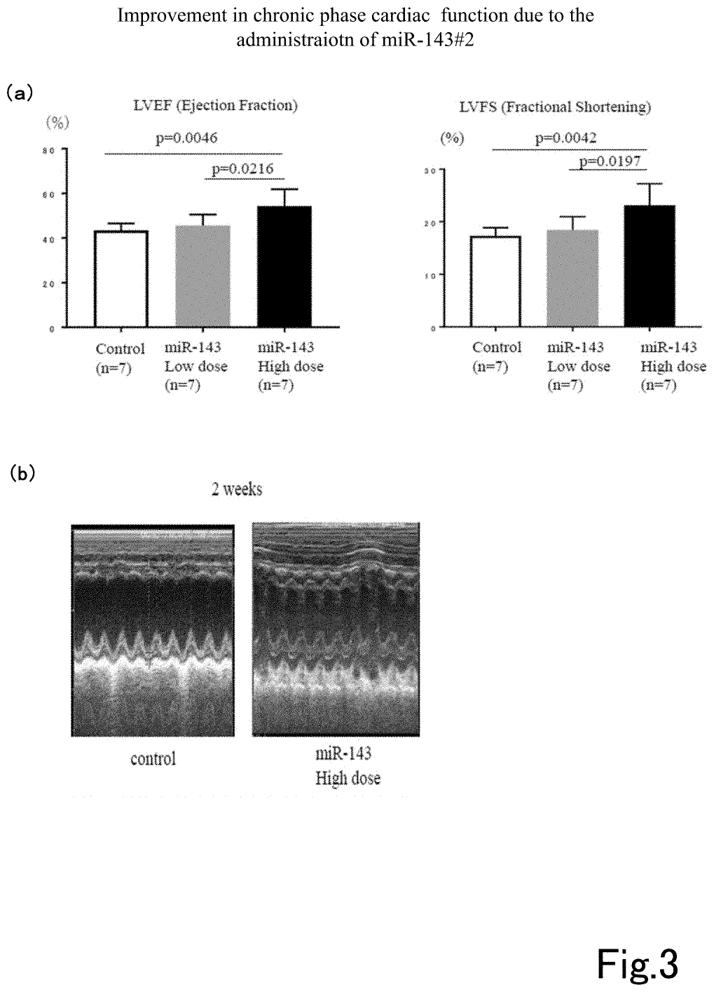

In a rat myocardial infarction model (reperfusion for 14 days after occlusion of the coronary artery for 30 minutes), miR-143 #12 with the composition given below was encapsulated in liposome and administered intravenously a single time (low dose: 3 μg/kg in 1 mL saline, high dose: 9 μg/kg in 1 mL saline) at 1 hour after the start of reperfusion. miR-143 #12, which is predicted to be more effective than commercially available miR-143 based on in vitro experiments, was selected for the animal experiments. miR-143 #12 is the miR-143 derivative designated as the SEQ-12 that is disclosed in WO 2017/179660. The sense strand and antisense strand of miR-143 #12 correspond to SEQ ID NOs: 3 and 4. LipofectamineRNAiMax (Invitrogen) was used as the liposome, and, after incubation in 200 μL of OPTI-MEM for 10 minutes, the solution for administration was prepared using physiological saline so that the total volume was 1 ml. The results are shown in FIG. 3.

| [C1] | |
|---|---|
| miR-143 | S: 3′-GGUCUCUACGUCGUGACGUGGAGU-5′ |
| Wild | AS: 5′-UGAGAUGAAGCACUGUAGCUCAGG-3′ |
| miR-143#12 | S: 3′-GGU C UCUACGUCGUGACGUGGAGU-5′ |
| | AS: 5′-U^{∧}G^{∧}AGAUGAAGCACUGUAGCUCA^{∧}dT^{∧}dT-3′ |

Single underline: mismatch
Double underline: 2′-F RNA (OH group at the 2′-position of ribose is replaced by fluorine)
Bold character: 2′-Ome RNA (OH group at the 2′-position of ribose is replaced by the methoxy group)
\hat{ }: PS (phosphorothioate linkage)

As shown in FIG. 3(a), LVEF and LVFS were improved in correspondence to the amount of administration of miR-143. In addition, as shown in FIG. 3(b), improvement in left ventricular wall motion was also demonstrated by echocardiogram. [Example 3]

(Effect of miR-143 #12 on Rat H9C2 Cardiomyocytes after Exposure to Hydrogen Peroxide)

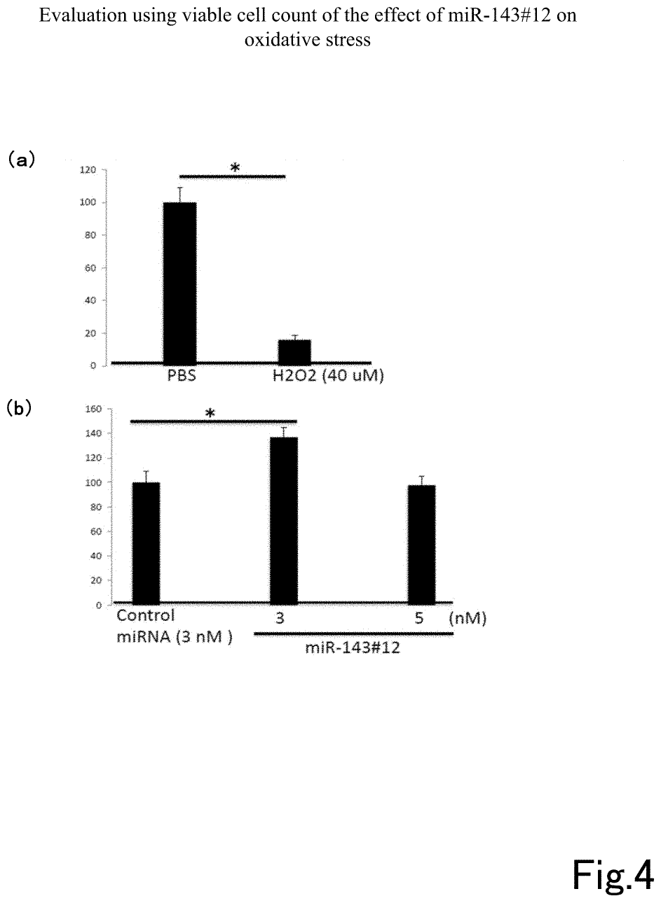

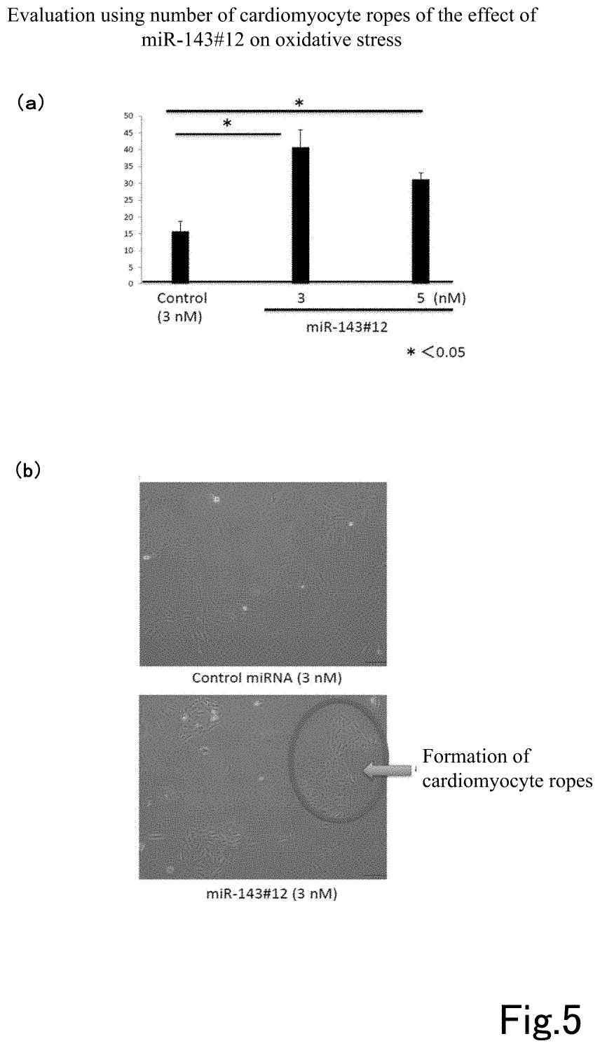

Rat cardiomyocytes were treated with PBS and a 40 μM hydrogen peroxide solution for 30 minutes. The medium was then exchanged and after 3 days the viable cell count was evaluated using trypan blue staining. The results are given in FIG. 4(a). In addition, rat cardiomyocytes were treated with a 40 μM hydrogen peroxide solution for 30 minutes, immediately followed by culture medium exchange and transfection with liposome-encapsulated miR-143 #12. After 5 days, the viable cell count was evaluated using trypan blue staining. 3 μL of Lipofectamine RNAiMax (Invitrogen) was used for the liposome, and, after incubation with 200 μL of OPTI-MEM, the transfection solution was added to provide 3 nM or 5 nM miR-143 #12 in 1 mL of culture medium per 1 well. As a control, the same procedure was performed using a control miRNA (S strand: 5′-UGAGGAGUAGUGAAAGGCC dtdt-3′ (SEQ ID NO: 8), AS strand: 5′-GGCCUUUCACUACUCCUCA dtdt-3′ (SEQ ID NO: 9)) that does not hybridize with the human genome. The results are given in FIG. 4(b).

As shown in FIGS. 4(a) and 4(b), the cardiomyocytes were damaged by exposure to hydrogen peroxide, but miR-143 #12 was shown not only to protect the cardiomyocytes from hydrogen peroxide-induced oxidative stress damage, but to also promote cell proliferation.

### Example 4

(Other Effects of miR-143 on Rat H9C2 Cardiomyocytes after Exposure to Hydrogen Peroxide)

Rat cardiomyocytes were treated for 30 minutes with a 40 μM hydrogen peroxide solution, followed by exchange of the culture medium and execution of the same procedure as in Example 3 to carry out transfection with liposome-encapsulated miR-143 #12. The cells were observed with a microscope after 5 days (after 120 hours), and the rope-forming colonies were counted per 200 colonies. The results are given in FIG. 5.

As shown in FIG. 5, it was found that miR-143 #12 opposed hydrogen peroxide-induced oxidative stress damage of the cardiomyocytes and promoted the formation of rope-like cardiomyocyte masses.

### Example 5

(Evaluation of the Administration of miR-143 #12 in a Rat Acute Myocardial Infarction Model)

(Influence of miR-143 #12 on Infarct Size)

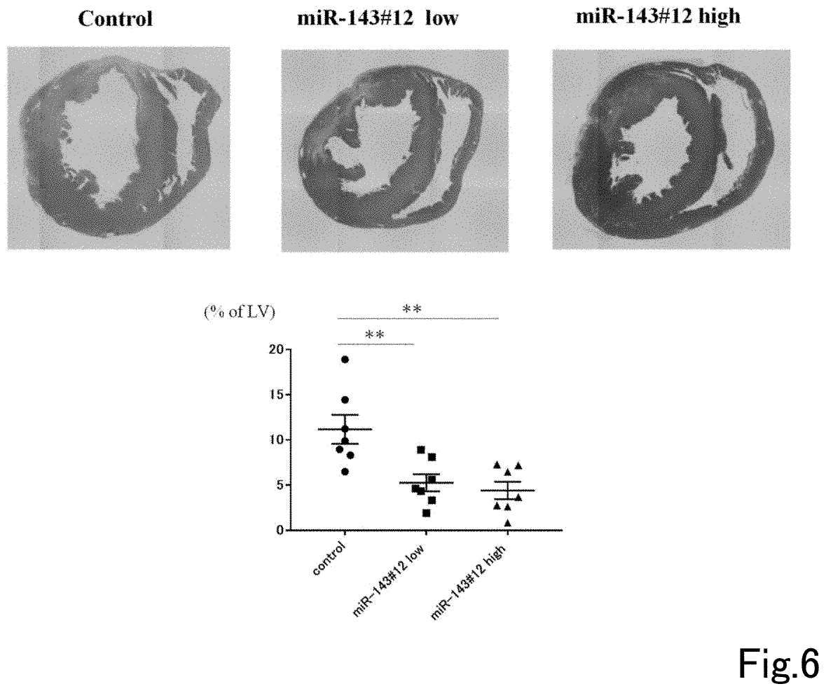

In a rat acute myocardial infarction model (reperfusion for 14 days after occlusion of the coronary artery for 30 minutes), miR-143 #12 with the composition given below was encapsulated in liposome and administered intravenously a single time (low dose: 3 μg/kg in 1 mL physiological saline, high dose: 9 μg/kg in 1 mL physiological saline) at 1 hour after the start of reperfusion. As in Example 2, miR-143 #12 was used as the miR-143. Lipofectamine RNAiMax (Invitrogen) was used as the liposome, and, after incubation in 200 μL of OPTI-MEM for 10 minutes, the solution for administration was prepared using physiological saline so that the aforementioned total volume was 1 mL. After 14 days, the hearts were excised from the rats, fixed in formalin, and embedded in paraffin, followed by the preparation of slides of 4 μm-thick sections and staining with Masson's trichrome to enable differentiation of infarct regions from non-infarct regions. After staining, the slide-mounted section was observed with an optical microscope and the infarct size was evaluated. The results are given in FIG. 6.

As shown in FIG. 6, the infarct size was significantly smaller in rats receiving the low dose of miR-143 #12 and in rats receiving the high dose of miR-143 #12 than in the control. It was also demonstrated that the infarct size declines in correspondence to the dose. Based on this, it was shown that the myocardial infarct size can be effectively reduced by the administration of miR-143 #12.

(Effect of miR-143 #12 on CD31-Positive Microvessels and α-Smooth Muscle-Positive Vessels)

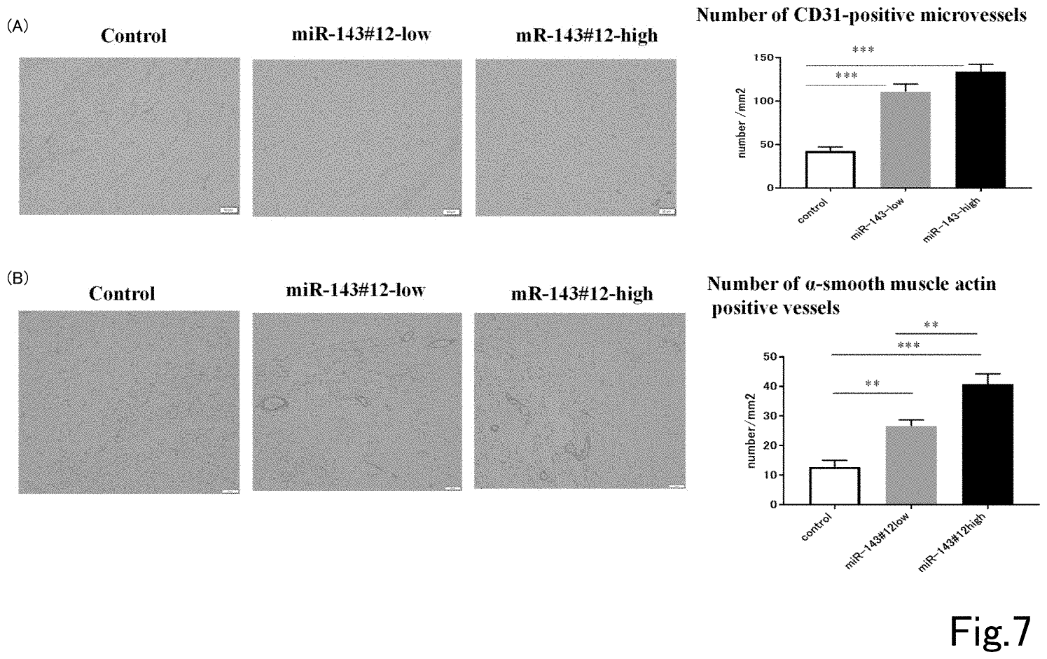

FIG. 7(A) and FIG. 7(B) give, respectively, the results of immunohistochemical staining, using anti-CD31 rabbit monoclonal antibody and anti-a-smooth muscle actin rabbit antibody, of tissue sections prepared from the aforementioned hearts that had been excised from the rats.

As shown in FIG. 7(A), the number of CD31-positive microvessels was increased, relative to the control, in rats receiving the low dose of miR-143 #12 and in rats receiving the high dose of miR-143 #12. As shown in FIG. 7(B), the number of α-smooth muscle-positive vessels, which have vascular smooth muscle, was increased in rats receiving the low dose of miR-143 #12 and in rats receiving the high dose of miR-143 #12. Based on the preceding, it was shown that miR-143 #12 caused the new formation of capillaries lacking vascular smooth muscle and of relatively thick blood vessels having vascular smooth muscle cells.

(Effects of miR-143 #12 on Cardiomyocyte Apoptosis)

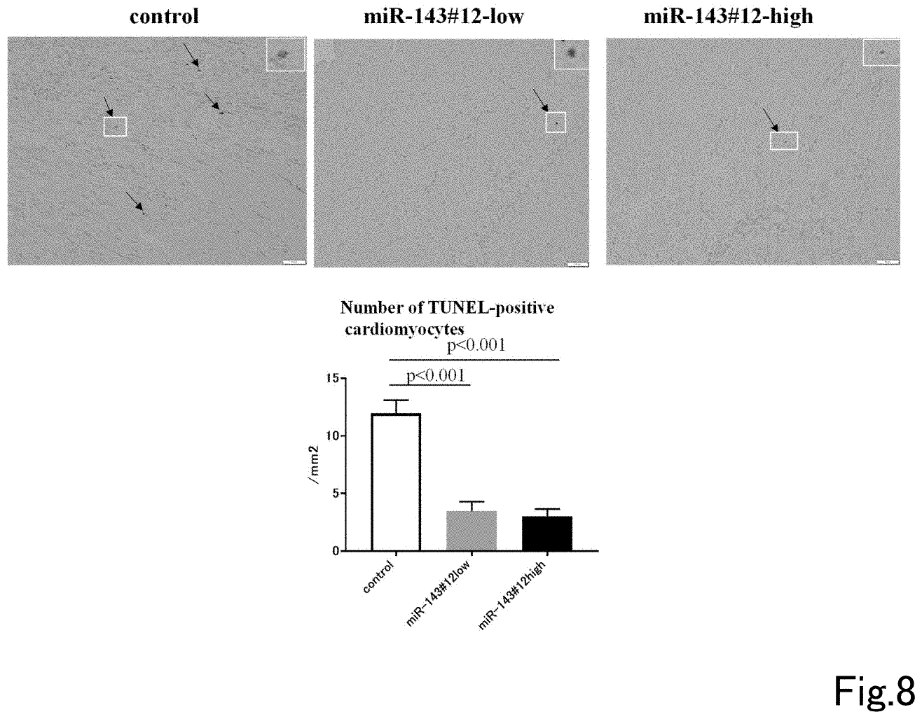

Apoptosis was detected by the TUNEL method in tissue sections prepared from the aforementioned hearts that had been excised from the rats. The results are given in FIG. 8. As shown in FIG. 8, there was a decline, in comparison to the control, in TUNEL-stained cardiomyocytes in rats receiving the low dose of miR-143 #12 and in rats receiving the high dose of miR-143 #12. That is, since there was a decline in cardiomyocytes entering apoptosis, it was shown that miR-143 #12 suppressed the entry of cardiomyocytes into apoptosis.

(Effect of miR-143 #12 on Cardiomyocyte Proliferation)

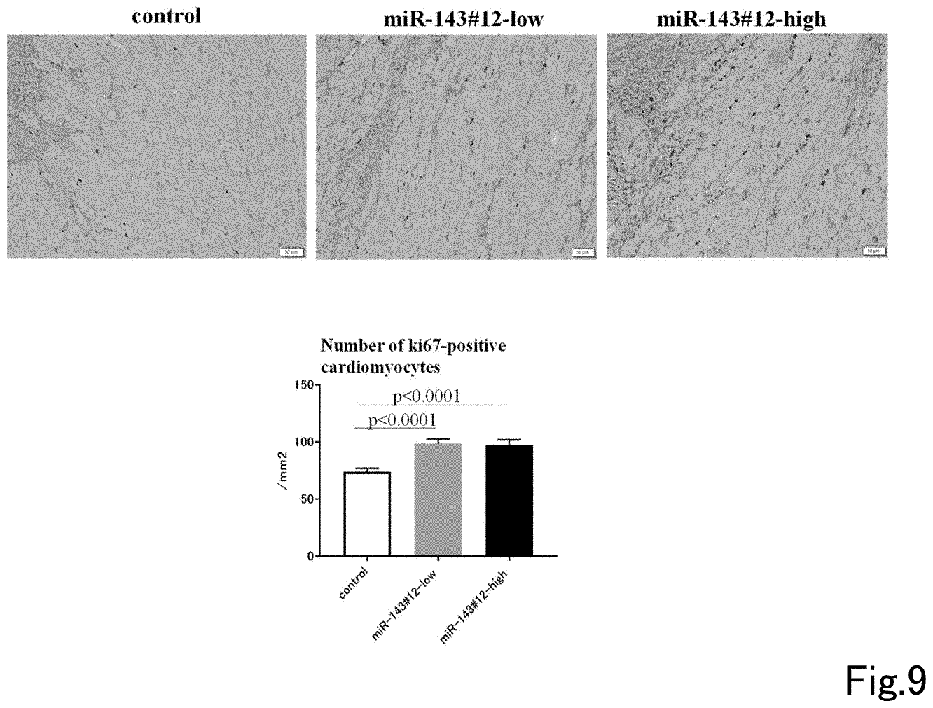

Tissue immunostaining was performed using anti-ki67 antibody on tissue sections prepared from the aforementioned hearts that had been excised from the rats. The results are given in FIG. 9. As shown in FIG. 9, in comparison to the control, the number of ki67-positive cardiomyocytes in the myocardial infarct border zone was significantly increased in rats receiving the low dose of miR-143 #12 and in rats receiving the high dose of miR-143 #12. Since ki67 is a marker indicative of cell proliferation, it was thus shown that miR-143 #12 induces cardiomyocyte proliferation.

### Example 6

(Effect of miR-143 #12 on Angiogenic Capability Using HUVEC (Human Umbilical Vein Endothelial Cells))

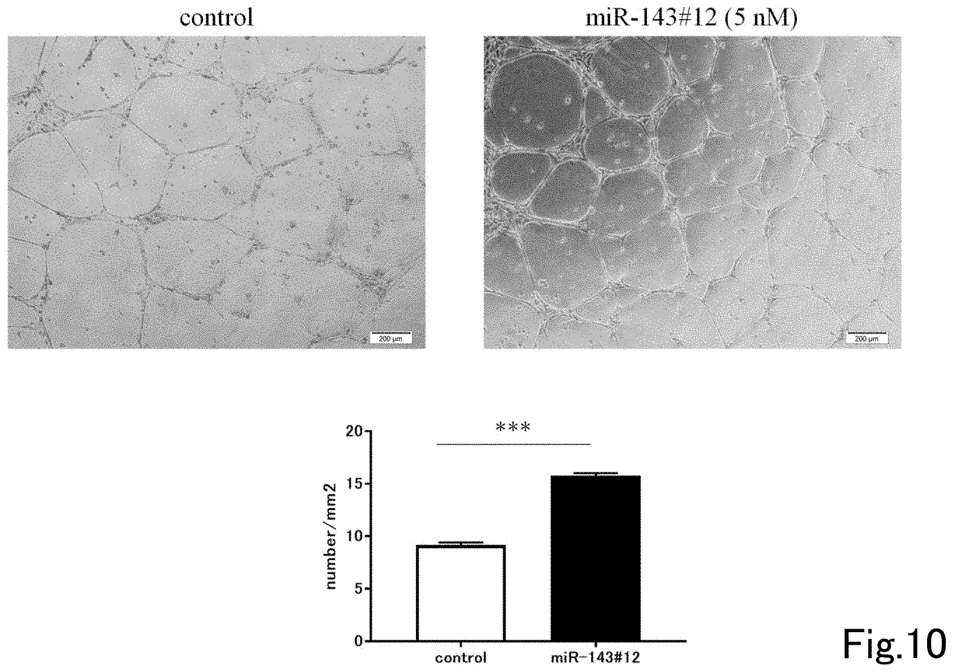

HUVEC (human umbilical vein endothelial cells) cells were seeded to a 6-well plate of the BD BioCoat Angiogenesis System: Endothelial Cell Tube Formation, and transfection was subsequently performed with liposome-encapsulated miR-143 #12. For transfection, 3 μL of Lipofectamine RNAiMax (Invitrogen) was used as the liposome, and, after incubation in 200 μL of OPTI-MEM for 10 minutes, the transfection solution was added to provide miR-143 #12 at 5 nM in 1 mL of culture medium per 1 well. The control used the same control miRNA as used in Example 3. The results are given in FIG. 10.

As shown in FIG. 10, as compared to the control, the number of lumens formed of the HUVEC cells was significantly increased in the group that received miR143 #12 (5 nM). That is, miR-143 #12 was shown to have the potential of promoting the angiogenic capability of vascular endothelial cells.

### Example 7

(Evaluation of Various miR-143 Derivatives in Rat H9C2 Cardiomyocytes)

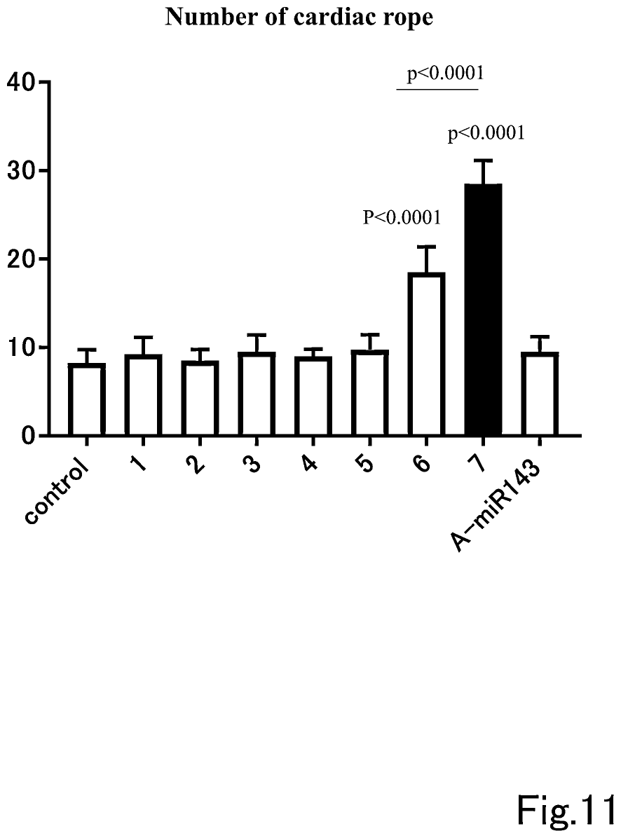

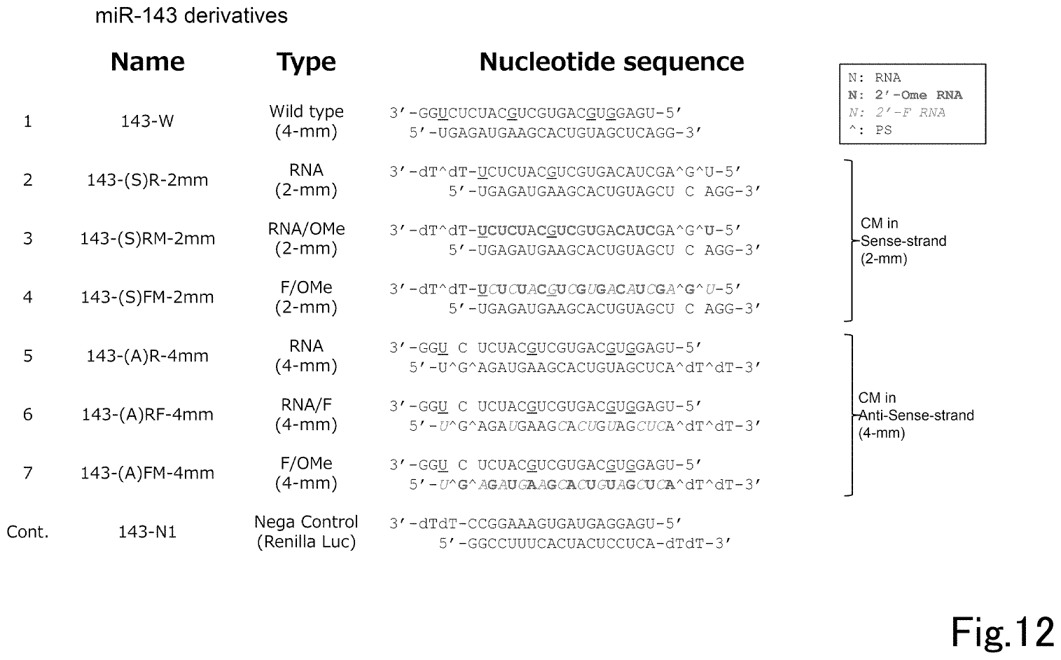

Rat H9C2 cardiomyocytes were exposed for 30 minutes to hydrogen peroxide (40 μM) in accordance with Examples 3 and 4, followed by the introduction of wild-type miR-143 (miRNA number 1 in FIGS. 11 and 12), 6 types of miR-143 derivatives including miR-143 #12 (miRNA number 7 in FIGS. 11 and 12), or A-miR143 (acquired from Ambion, Inc.). The cells were observed with a microscope after 5 days (after 120 hours), and the rope-forming colonies were counted per 200 colonies. The results are given in FIG. 11. FIG. 12 shows the details of the miR-143s that were used. In FIG. 12, mm indicates a mismatch; “4-mm” indicates 4 mismatches; and “2-mm” indicates 2 mismatches. The position of a mismatch is indicated by an underline. A “A” indicates that the internucleotide linkage is a phosphorothioate linkage; a bold character denotes a ribonucleotide in which a methoxy group is provided in place of the hydroxyl group at the 2′-position of the ribonucleotide; and an italicized character indicates a ribonucleotide in which the F atom is provided in place of the hydroxyl group at the 2′-position of the ribonucleotide.

As shown in FIG. 11, miR-143 #12 exhibited the highest cardiac rope formation capability. This result suggests that miR-143 #12 induces reprogramming in injured cardiomyocytes, bringing about differentiation and a proliferation capability.

Based on the preceding, it was found that miR-143 has the effect of shrinking the size of myocardial infarction, with the mechanisms for this being an angiogenic activity, myocardial apoptosis inhibitory activity, and cardiomyocyte proliferation activity (myocardial regeneration activity).

### Sequence Listing Free Text

SEQ ID NOs: 3-5: miR-143 derivative
SEQ ID NOs: 8 and 9: control miRNA

### [Sequence Listing]

### CITATION LIST

Non Patent Literature 1: Higashi K, Akao Y & Minatoguchi S et al. MicroRNA-145 Repairs Infarcted Myocardium by Accelerating Cardiomyocyte Autophagy. Am J Physiol Heart Circ Physiol, 309 (11), H1813-26, 2015

1. A method for assessing a chronic phase cardiac function in acute myocardial infarction, comprising monitoring blood concentration of miR-143 and/or miR-145 in an acute phase of the acute myocardial infarction in an individual who has occurred the acute myocardial infarction or presents a possibility of occurrence of the acute myocardial infarction.

2. The method according to claim 1, wherein the monitoring is performed in any interval within two weeks from immediately after the occurrence of the acute myocardial infarction in the individual.

3. The method according to claim 1, wherein the monitoring estimates chronic phase cardiac function of the individual based on an amount of change over time in the blood concentration.

4. The method according to claim 1, wherein the monitoring estimates a favorable chronic phase cardiac function for the individual when the blood concentration increases over time.

5. The method according to claim 1, wherein the monitoring estimates a poor chronic phase cardiac function for the individual when when the blood concentration does not increase over time.

6. The method according to claim 1, wherein the monitoring compares the amount of change over time in the blood concentration with a reference value that can estimate the chronic phase cardiac function of the individual.

7. An apparatus of predicting a chronic phase cardiac function for an acute myocardial infarction, the apparatus being configured to acquire an amount of change over time in an acute phase blood concentration of miR-143 and/or miR-145 acquired for an individual who has occurred the acute myocardial infarction or presents a possibility of occurrence of the acute myocardial infarction.

8. The apparatus according to claim 7, wherein the apparatus predicts the chronic phase cardiac function by comparing the amount of change over time with a preliminarily acquired reference value that can predict the chronic phase cardiac function of the individual.

9. A system of predicting a chronic phase cardiac function for an acute myocardial infarction, the system comprising a means of acquiring an amount of change over time in acute phase blood concentration of miR-143 and/or miR-145 acquired for an individual who has occurred the acute myocardial infarction or presents a possibility of occurrence of the acute myocardial infarction.

10. The system according to claim 9, the system further comprising a means of performing a comparison of the amount of change over time with a preliminarily acquired reference value that can predict the chronic phase cardiac function of the individual and that performs a prediction of the chronic phase cardiac function, based on results of the comparison.

11. A drug for improving or treating a chronic phase cardiac function after acute myocardial infarction, comprising miR-143 and/or miR-145 or a compound that acts like miR-143 and/or miR-145 in an individual as an active ingredient thereof.

12. The drug according to claim 11, that is administered in an acute phase after an acute myocardial infarction.

13. A drug for improving or treating acute myocardial infarction, comprising miR-143 and/or miR-145 or a compound that acts like miR-143 and/or miR-145 in an individual as an active ingredient thereof.

14. A test kit of predicting a chronic phase cardiac function after acute myocardial infarction, wherein the kit comprises a reagent that specifically detects miR-143 and/or miR-145.


SEQUENCE LISTING (9 sequences)

SEQ ID NO: 1 - RNA, 22 nt, Homo sapiens
ggugcagugcugcaucucuggu

SEQ ID NO: 2 - RNA, 21 nt, Homo sapiens
ugagaugaagcacuguagcuc

SEQ ID NO: 3 - RNA, 24 nt, Artificial sequence
  Passenger strand of miR-143 derivative
ugaggugcagugcugcaucucugg

SEQ ID NO: 4 - RNA, 22 nt, Artificial sequence
  Guide strand of miR-143 derivative
ugagaugaagcacuguagcuca

SEQ ID NO: 5 - RNA, 24 nt, Artificial sequence
  Guide strand of miR-143 derivative
ugagaugaagcacuguagcucagg

SEQ ID NO: 6 - RNA, 23 nt, Homo sapiens
guccaguuuucccaggaaucccu

SEQ ID NO: 7 - RNA, 22 nt, Homo sapiens
ggauuccuggaaauacuguucu

SEQ ID NO: 8 - RNA, 19 nt, Artificial sequence
  Contrl miRNA
ugaggaguagugaaaggcc

SEQ ID NO: 9 - RNA, 19 nt, Artificial sequence
  Control miRNA
ggccuuucacuacuccuca